(19) United States
(12) Patent Application Publication
(10) Pub. No.: US 2025/0325811 A1
(43) Pub. Date: Oct. 23, 2025

(54) VESTIBULAR STIMULATION FOR TREATMENT OF MOTOR DISORDERS

(71) Applicant: UNIVERSIDAD DE LAS PALMAS DE GRAN CANARIA, LAS PALMAS DE GRAN CANARIA (ES)

(72) Inventor: Angel Ramos de Miguel, Las Palmas (ES)

(21) Appl. No.: 18/864,617

(22) PCT Filed: Apr. 28, 2023
(86) PCT No.: PCT/EP2023/061229

§ 371 Date: Nov. 11, 2024

(30) Foreign Application Priority Data
May 18, 2022 (EP) 22382475.6

(51) Int. Cl. A61N 1/36; A61N 1/02; A61N 1/05

(52) U.S. Cl. CPC A61N 1/36067; A61N 1/025; A61N 1/0541; A61N 1/36003; A61N 1/36036; A61N 1/3614; A61N 1/36178

(57) ABSTRACT

Presented herein are techniques for specifically electrically stimulating a recipient's vestibular system in order to treat motor disorders. such as ataxia or Parkinson's Disease. More specifically. in accordance with embodiments presented herein, one or more electrodes are implanted in a recipient so as to deliver electrical stimulation (e.g., current) signals to a portion of the vestibular in a manner that remediates motor disorder symptoms experienced by the recipient.

### BACKGROUND

### Field of the Invention

The present invention generally relates to treatment of motor disorders, such as Parkinson's disease or ataxia, via vestibular stimulation.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: assessing fine motor skills of a recipient of an implantable motor disorder stimulator device; and delivering, with the implantable motor disorder stimulator device, electrical stimulation signals to a vestibular system of the recipient, wherein one or more parameters of the electrical stimulation signals are set based on the assessing of the fine motor skills of the recipient.

In another aspect, a method is provided. The method comprises: sensing at least one of hand or head tremors of a recipient; and delivering electrical stimulation signals to a vestibular system of the recipient based on the sensing of the at least one of the hand tremors or head tremors of the recipient.

In another aspect, one or more non-transitory computer readable storage media are provided. The one or more non-transitory computer readable storage media comprise instructions that, when executed by a processor, cause the processor to: deliver, via at least one electrode configured to be implanted at an inner ear of a recipient, electrical stimulation signals to a peripheral vestibular system of the inner ear; monitor one or more target motor disorder symptoms of the recipient following initiation of delivery of the electrical stimulation signals; and adjust one or more parameters of the electrical stimulation signals based on the monitoring of the one or more target motor disorder symptoms of the recipient.

In another aspect, an implantable motor disorder stimulator system is provided. The implantable motor disorder stimulator system comprises: one or more electrodes configured to be implanted at an inner ear of a recipient with a motor disorder; an implantable stimulator unit configured to deliver, via at least one of the one or more electrodes, electrical stimulation signals to a vestibular system of the inner ear of the recipient; and at least one processor configured to: following delivery of the electrical stimulation to a vestibular system, analyze one or more motor disorder symptoms experienced by the recipient, and adjust, based on the analyzing of the one or more motor disorder symptoms experienced based on the recipient, one or more parameters of the electrical stimulation signals to reduce the one or more motor disorder symptoms experienced by the recipient.

In another aspect, an implantable motor disorder stimulator is provided. The implantable motor disorder stimulator comprises: a stimulating assembly comprising a plurality of stimulating elements configured to be implanted in an inner ear of a recipient adjacent to the otolith organs of the inner ear; and a stimulator unit configured to generate and deliver electrical stimulation signals to the recipient's vestibular nerve of via one or more of the otolith organs, wherein the electrical stimulation signals have stimulation parameters determined based on one or more assessments of the recipient's fine motor skills.

### BRIEF DESCRIPTION OF THE DRAWINGS

Embodiments of the present invention are described herein in conjunction with the accompanying drawings, in which:

FIG. 1A is a schematic, partial cross-sectional view illustrating anatomical structures of the human inner ear;

FIG. 1B is a perspective view illustrating further details of a portion of the human inner ear of FIG. 1A;

FIG. 2A is a schematic diagram illustrating a otolith stimulation system, in accordance with certain embodiments presented herein;

FIG. 2B is a simplified block diagram of the vestibular stimulation system of FIG. 2A, in accordance with certain embodiments presented herein;

FIG. 3A is an image illustrating implantation of a stimulating assembly of an implantable motor disorder stimulator into a recipient, in accordance with certain embodiments presented herein;

FIGS. 3B, 3C, 3D, 3E, 3F, and 3G are annotated computerized tomography (CT) scans illustrating implantation of a stimulating assembly of an implantable motor disorder stimulator into a recipient, in accordance with certain embodiments presented herein;

FIG. 3H is a medical image illustrating nerve cells adjacent to an implanted location of a stimulating assembly of an implantable motor disorder stimulator, in accordance with certain embodiments presented herein;

FIG. 4 is a schematic three-dimensional diagram of a recipient's inner ear having a stimulating assembly implanted therein, in accordance with certain embodiments presented herein;

FIG. 5 is a flowchart of a method, in accordance with certain embodiments presented herein;

FIG. 6 is a flowchart of a method, in accordance with certain embodiments presented herein;

FIG. 7 is a schematic diagram illustrating a fitting system with which aspects of the techniques presented herein can be implemented;

FIG. 8 is a flowchart of another method, in accordance with certain embodiments presented herein;

FIG. 9 is a flowchart of another method, in accordance with certain embodiments presented herein; and

FIG. 10 is a flowchart of another method, in accordance with certain embodiments presented herein.

### DETAILED DESCRIPTION

There are number of different types of motor disorders that cause unintended or uncontrollable movements of the body. For example, ataxia is one of the categories of neurological diseases/conditions which can affect a patients' limb coordination, speech function, eye movement, and muscle control at different levels. Ataxia occurs when the part of the brain called the cerebellum is damaged. There are several types of ataxia, including: ataxia telangiectasia (AT), episodic ataxia, Friedreich's ataxia, multiple system atrophy (MSA) and spinocerebellar ataxia This condition happens when the part of the brain called the cerebellum is damaged

Parkinson's Disease (Parkinson's or PD) is another neurological disease and occurs when nerve cells (neurons) in an area of the brain called the substantia nigra become impaired or die. These cells normally produce dopamine, a chemical (neurotransmitter) that helps the cells of the brain communicate (transmits signals, “messages,” between areas in the brain). When these nerve cells become impaired or die, they produce less dopamine. Dopamine is especially important for the operation of another area of the brain called the basal ganglia. This area of the brain is responsible for organizing the brain's commands for body movement. The loss of dopamine causes the movement symptoms seen in patients with Parkinson's disease.

Patients with Parkinson's disease also lose another neurotransmitter called norepinephrine. This chemical is needed for proper functioning of the sympathetic nervous system. This system controls some of the body's autonomic functions such as digestion, heart rate, blood pressure and breathing. Loss of norepinephrine causes some of the non-movement-related symptoms of Parkinson's disease.

Essential tremor is another disorder of the nervous system that causes a rhythmic shaking or tremor. It can affect almost any part of the body but the trembling most often occurs in the hands and is especially bothersome during the attempt to do simple tasks like drinking from a glass or writing with a pencil. Essential tremor may also affect one's head, voice, arms, or legs. While it is not the same as Parkinson's, the tremor of Parkinson's resembles essential tremor.

The signs and symptoms of motor disorders can vary. For example, patients with ataxia lose muscle control in their arms and legs, which may lead to a lack of balance, coordination, and trouble walking. Ataxia may affect the fingers, hands, arms, legs, body, speech, and even eye movement. The symptoms of Parkinson's can generally be divided into motor and nonmotor. Motor symptoms are those that affect movement of the body. These are the most obvious symptoms of the disease. The main motor symptoms of Parkinson's are tremors (e.g., head and/or hand tremors), slowness of movement (called “bradykinesia”), stiffness (“rigidity”), and poor balance (“postural instability” or “gait impairment”). These symptoms are usually mild in the early stages of the disease. The symptom of tremor caused by Parkinson's disease is the most noticeable when a person is at rest. The tremor of early Parkinson's is intermittent and may not be noticeable to others. Tremor usually becomes noticeable one hand at a time, spreading to the second hand over a period of a few years.

Most motor disorders progressively worsen over time, although the rate varies greatly from person to person. A number of treatments are available to help manage the symptoms and improve a person's quality of life. However, there is no cure for these diseases at this time.

Presented herein are techniques for specifically electrically stimulating a recipient's vestibular system in order to treat motor disorders, such as ataxia or Parkinson's. More specifically, in accordance with embodiments presented herein, one or more electrodes are implanted in a recipient so as to deliver electrical stimulation (e.g., current) signals to a portion of the vestibular in a manner that remediates motor disorder symptoms experienced by the recipient. In general, the vestibular stimulation presented herein can be delivered from/via one or electrodes implanted within (inside) the inner ear, or from/via one or more electrodes disposed at outer surface of the inner ear (e.g., implanted adjacent the inner ear).

Merely for ease of description, the techniques presented herein are primarily described with reference to a specific implantable medical device system, namely a stand-alone vestibular stimulation system. However, it is to be appreciated that the techniques presented herein may also be partially or fully implemented by other types of implantable medical devices, the techniques presented herein may be implemented by auditory prosthesis systems that include one or more other types of auditory prostheses, such as cochlear implants, middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

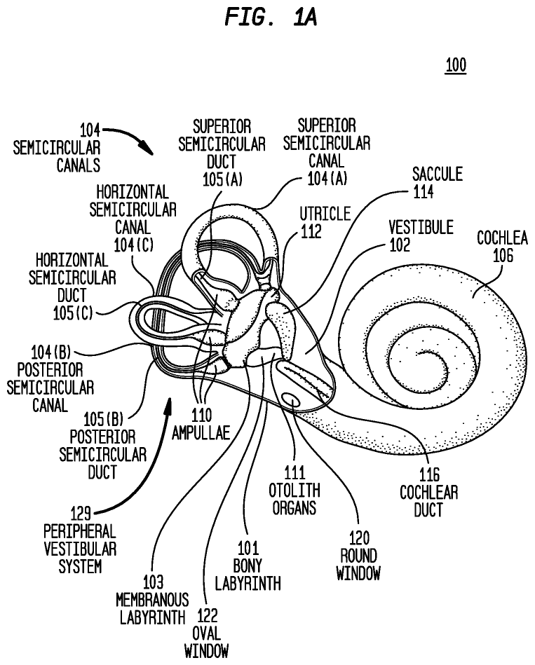

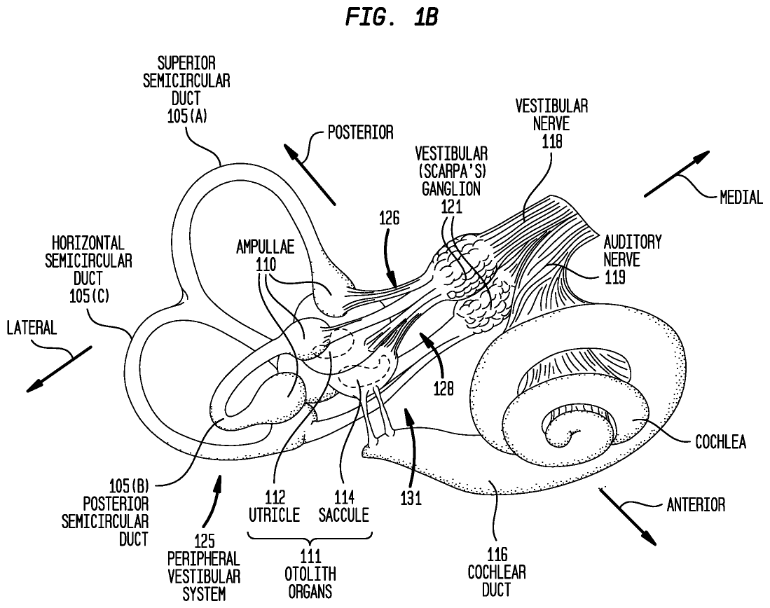

Before describing details of the techniques presented herein, relevant aspects of an example human inner ear are first described below with reference to FIGS. 1A and 1B. In particular, shown in FIG. 1A is the bony labyrinth 101, which is the bony outer wall of an inner ear 100. The bony labyrinth 101 includes three sections/parts, referred to as the vestibule 102, which includes the Otolith organs 111, the semicircular canals 104, and the cochlea 106. The vestibule 102, the semicircular canals 104, and the cochlea 106 are cavities that are internally lined with periosteum and that contain a fluid known as perilymph. For ease of illustration, a portion of the bony labyrinth 101 forming the vestibule 102 has been omitted from FIG. 1A, while the entire bony labyrinth 101 has been omitted from FIG. 1B.

Within the bony labyrinth 101 is the membranous labyrinth 103, which consists of the semicircular ducts 105, the otolith organs 111 (i.e., the utricle 112 and the saccule 114), and the cochlear duct 116. The membranous labyrinth 103 is filled with a fluid known as endolymph, and is surrounded by the perilymph of the bony labyrinth 101. The membranous labyrinth 103 is also suspended from the bony labyrinth 101 by fine connective tissue strands.

As shown, the bony labyrinth 101 includes three (3) semicircular canals 104, referred to as the superior or anterior semicircular canal 104(A), the posterior semicircular canal 104(B), and the horizontal or lateral semicircular canal 104(C). Within the superior semicircular canal 104(A) is the superior semicircular duct 105(B), within the posterior semicircular canal 104(B) is the posterior semicircular duct 105(B), and within the horizontal semicircular canal 104(C) is the horizontal semicircular duct 105(C). The semicircular ducts 105 are situated superoposterior to the vestibule 102 and each have a swelling at one end, known as an ampulla 110 (i.e., three ampullae are shown in FIGS. 1A and 1B, one for each duct 105). The semicircular ducts 105, the utricle 112, and the saccule 114 are sometimes collectively referred to as the “peripheral vestibular apparatus” or the “peripheral vestibular system” 125.

The semicircular ducts 105(A), 105 (B), and 105(C) are half-circular, interconnected tubes that are aligned approximately orthogonally to one another (i.e., at right angles to each other) so that they measure motions in all three planes. Specifically, lateral duct 105(C) is aligned roughly horizontally in the head, while the superior 105(A) and posterior ducts 105(B) are aligned roughly at a 45 degree angle to a vertical through the center of the individual's head. The semicircular ducts 105(A), 105(B), and 105(C) are each maximally sensitive to angular accelerations (head rotations) that lie in the plane of the duct. The result of this arrangement is that three semicircular ducts 105(A), 105(B), and 105(C) can uniquely specify the direction and amplitude of any arbitrary head rotation. That is, upon movement of the head, the flow of endolymph within the ducts 105 changes speed and/or direction. Sensory receptors in the ampullae 110 detect these changes, and send signals to the brain via the vestibular nerve 118 (FIG. 1B), allowing for the processing of balance.

As noted, the membranous labyrinth 103 also includes the utricle 112 and the saccule 114, which are collectively referred to as the otolith organs 111. The utricle 112 and the saccule 114 are two membranous sacs located in the vestibule 102, which detect movement or acceleration of the head in the horizontal and vertical planes, respectively (i.e., linear accelerations). The utricle 112 is the larger of the two, receiving the three semi-circular ducts 105. The saccule 114 is globular in shape and receives the cochlear duct 116.

The utricle 112 and the saccule 114 each contain a macula, which is an organ consisting of a patch of hair cells covered by a gelatinous membrane containing particles of calcium carbonate, called otoliths. Motions of the head cause the otoliths organs 111 to pull on these hair cells, stimulating the vestibular nerve 118, which allow the individual to perceive linear acceleration, both horizontally and vertically, and gravity control (i.e., gravitoinertial information).

The vestibular nerve 118 is one of the two branches of the vestibulocochlear nerve (the other being the auditory nerve 119), which functions to relay/transmit sensory information transmitted by the vestibular hair cells located in the two otolith organs (i.e., the utricle 112 and the saccule 114) and the three semicircular ducts 105 via the vestibular ganglion 121. Again, as noted, information from the otolith organs 111 reflects gravity and linear accelerations of the head, while information from the semicircular ducts 105 reflects rotational movement of the head.

The peripheral vestibular nerve fibers are generally divided into three branches. First, the superior vestibular nerve branch 126 passes through the foramina in the area vestibularis superior and ends in the utricle 112 and in the ampullae 110 of the superior and horizontal semicircular ducts 105(A) and 105(C), respectively. Second, the inferior vestibular nerve branch 128 traverse the foramina in the area vestibularis inferior and ends in the saccule 114. Third, posterior vestibular nerve branch 131 runs through the foramen singulare and supplies the ampulla 110 of the posterior semicircular duct 105(B), in more than 50% of the cases is part of the inferior branch.

Also shown in FIG. 1A is the round window 120 and the oval window 122. The round window 120 and oval window 122 are the two openings from the middle ear (not shown) into the inner ear 100. The round window 120 is situated inferior to (below) and posterior to (behind) the oval window 122, from which it is separated by the promontory (rounded elevation). The oval window 122 is sealed by a membrane (oval window membrane) and leads from the middle ear to the vestibule of the inner ear 100. Vibrations that contact the tympanic membrane (ear drum) in the outer ear (not shown) travel through the three ossicles (i.e., malleus, incus, and stapes) of the middle ear and into the inner ear 100 via the oval window 122. That is, the oval window 122 is the intersection of the middle ear with the inner ear 100 and is directly contacted by the stapes. The round window 120 is also sealed by a membrane (round window membrane), which vibrates with opposite phase to vibrations entering the inner ear 100 through the oval window 122. The round window 120 allows fluid in the cochlea 106 to move.

Presented herein are techniques for treating of motor disorders, such as ataxia or Parkinson's disease (Parkinson's or PD), via specific electrical stimulation of the vestibular system (e.g., stimulation of the sacculus, inferior vestibular nerve, etc.) from an implanted location, either within to adjacent to the inner ear. In accordance with embodiments presented herein, the neuro-stimulation system includes a stimulating assembly, which comprises one or more electrodes, that is configured to be implanted into the recipient (e.g., adjacent to the otolith organs). The stimulating assembly can be implanted, via, for example, the recipient's oval window, through an anterior opening such as an estapedotomy, etc. Once the stimulating assembly is implanted, the system is configured to specifically electrically stimulate the vestibular system in a manner that remediates symptoms (e.g., motor or nonmotor symptoms) associated with a motor disorder.

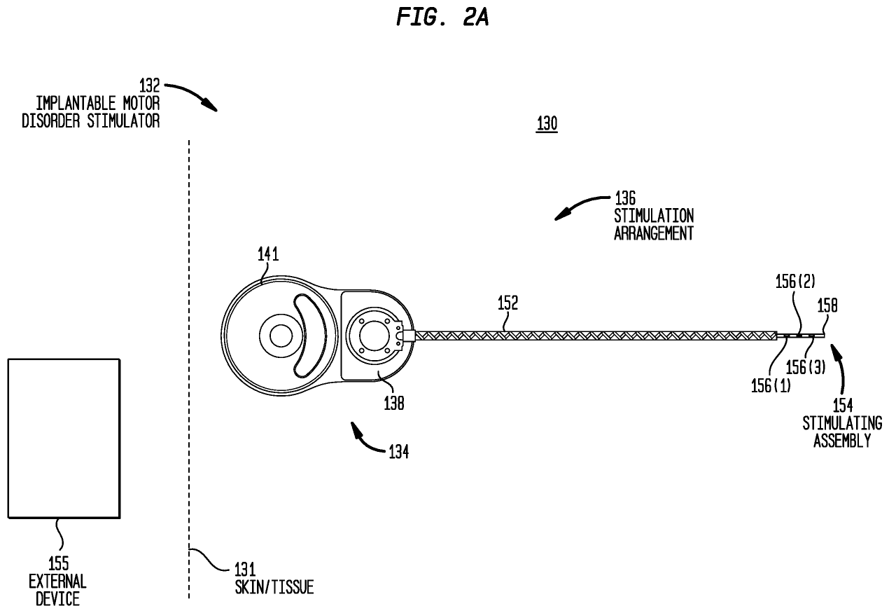

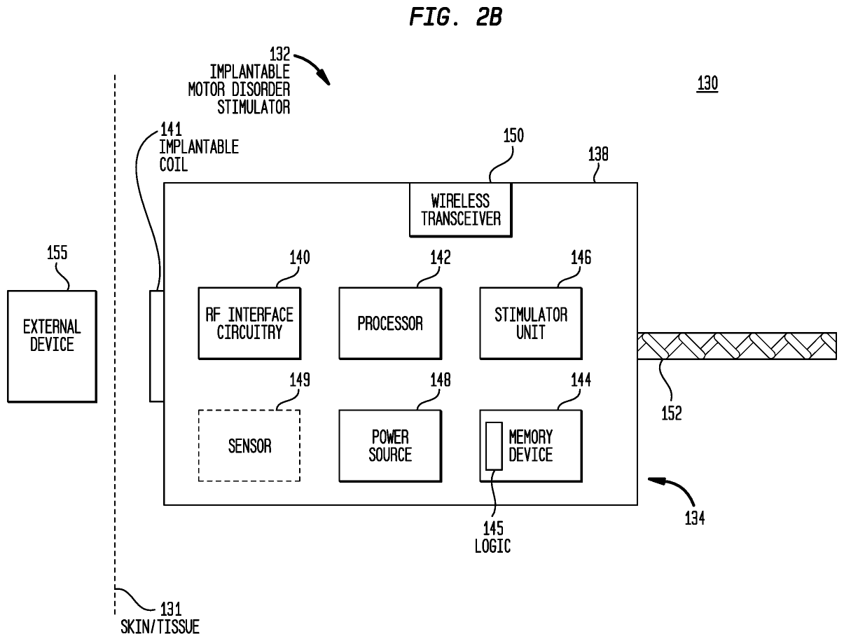

FIGS. 2A and 2B illustrate further details of one example neuro-stimulation system in accordance with embodiments presented herein. More specifically, shown in FIG. 2A is a perspective view of a motor disorder stimulation system 130, which includes an implantable motor disorder stimulator 132, while FIG. 2B is a block diagram of the implantable motor disorder stimulator 132. For ease of description, FIGS. 2A and 2B will be described together. Also for ease of illustration, certain components of the implantable motor disorder stimulator 132 are described with reference to the inner ear 100 of FIGS. 1A and 1B.

The implantable motor disorder stimulator 132 comprises an implant body (main module) 134 and a stimulation arrangement 136, both of which are implantable within a recipient (i.e., implanted under the skin/tissue 131 of a recipient). The implant body 134 generally comprises a hermetically-sealed housing 138 in which Radio-Frequency (RF) interface circuitry 140, at least one processor 142, a memory device (memory) 144 storing motor disorder remediation logic 145, a stimulator unit 146, a rechargeable power source 148, and a wireless transmitter/receiver (transceiver) 150 are disposed. The implant body 134 also includes an internal/implantable coil 141 that is generally external to the housing 138, but which is connected to the RF interface circuitry 140 via a hermetic feedthrough (not shown in FIG. 2B).

The processor 142 may be formed by one or more processors (e.g., one or more Digital Signal Processors (DSPs), one or more uC cores, etc.), firmware, software, etc. arranged to perform operations described herein. That is, the processor 142 may be implemented as firmware elements, partially or fully implemented with digital logic gates in one or more application-specific integrated circuits (ASICs), partially in software, etc. In general, the processor 142 may execute motor disorder remediation logic 145 and instruct the stimulator unit 146 to generate and deliver electrical stimulation signals to the recipient. The processor 142 may also perform other operations, include data logging, battery monitoring and low-battery alarm, etc. The stimulator unit 146 may include, for example, one or more current sources, switches, etc., that collectively operate to generate and deliver the electrical stimulation signals to the recipient via the stimulation arrangement 136.

As shown in FIG. 2A, the vestibular stimulation arrangement 124 comprises a lead 152 and a vestibular nerve stimulating (electrode) assembly 154. The stimulating assembly 154 comprises a plurality of stimulating elements 156 disposed in a carrier member 158 (e.g., a flexible silicone body). In this specific example, the stimulating assembly 154 comprises three (3) stimulating elements, and the stimulating elements comprise electrodes, referred to as electrodes 156(1), 156(2), and 156(3). As described further below, the electrodes 156(1), 156(2), and 156(3) function as an electrical interface to the recipient's vestibular system. It is to be appreciated that this specific embodiment with three electrodes is merely illustrative and that the techniques presented herein may be used with stimulating assemblies having different numbers of electrodes, stimulating assemblies having different lengths, etc.

As described elsewhere herein, the stimulating assembly 154 is configured such that a surgeon can implant the stimulating assembly adjacent the otolith organs 111 via, for example, the recipient's oval window 122. That is, the stimulating assembly 154 has sufficient stiffness and dynamics such that the stimulating assembly can be inserted through the oval window 122 and placed reliably within the bony labyrinth 101 adjacent the otolith organs 111 (e.g., sufficient stiffness to insert the stimulating assembly to the desired depth between the bony labyrinth 101 and the membranous labyrinth 103). In certain examples, the stimulating assembly 154 is configured to be placed adjacent the saccule 114.

The stimulating assembly 154 can have a stiffness allowing a single stroke atraumatic insertion to the required depth in the bone labyrinth 101. However, the stimulating assembly 154 may also have sufficient flexibility to deflect and avoid damage to the delicate anatomical structures of the inner ear 100. In addition, the lead 152 can have a configuration (e.g., length, flexibility, etc.) that allows for ease of surgical placement of the stimulating assembly 154 and that improves lead reliability (impact, fatigue, stress, etc.). In certain examples, the stimulating assembly 154 includes a removable or deformable stiffening member allowing placement of the stimulating assembly within the bony labyrinth 101.

As noted above, the implantable motor disorder stimulator 132 comprises RF interface circuitry 140 and a rechargeable power source 148 (e.g., one or more rechargeable batteries). The power source 148 can be recharged, for example, using power received from an external charger device via the RF interface circuitry 140. That is, although not shown in FIG. 2B, the external device 154 comprises an external coil configured to be inductively coupled with the implantable coil 141. When inductively coupled, the external coil and the implantable coil 141 form a closely-coupled wireless link by which power is transferred from a power source of the external device through the skin/tissue 131 of the recipient. In certain examples, the closely-coupled wireless link is a radio frequency (RF) link. However, various other types of energy transfer, such as infrared (IR), electromagnetic, capacitive and inductive transfer, may be used to transfer the power and/or data from the external device to the implantable motor disorder stimulator 132.

As described elsewhere herein, implantable motor disorder stimulators in accordance with certain embodiments presented herein do not necessarily need to rely on inputs from body motion sensors to deliver an effective treatment to the recipient. However, also shown in FIG. 2B is an optional one or more sensors 149 which may be used in certain embodiments. The one or more sensors 149 may include, for example, motion sensors such as accelerometers, gyroscopes, magnetometer, or sensor configured to, for example, to sense gravitoinertial accelerations (e.g., measure linear accelerations). In certain embodiments, the one or more motion sensors 149 are used to detect head shaking/head tremors, etc.

In accordance with certain embodiments, the implantable motor disorder stimulator 132 can operate with one or more external devices. FIGS. 2A and 2B illustrate one such example external device 155. In certain examples, at least one of the one or more external devices can include, for example, an external charger configured to provide power to the implantable motor disorder stimulator 132. In other examples, at least one of the one or more external devices can be configured to provide data to the implantable motor disorder stimulator 132 for use in delivering an effective treatment to the recipient. For example, at least one of the one or more external devices could be a mobile computing device (e.g., mobile phone) or a wearable device with one or more motion sensors (e.g., accelerometers, gyroscopes, magnetometer, etc.) configured to, for example, to sense kinematic data. In certain embodiments, the wearable device is configured to be worn on the wrist of the recipient (e.g., a smart watch) and can be used to detect hand shaking/hand tremors, while in other embodiments the wearable device is configured to worn at the head of the recipient (e.g., Personal Sound Amplification Products (PSAPS), earbuds etc.) and can be used to detect head shaking/hand tremors. Multiple external devices can be used with the implantable motor disorder stimulator 132 at the same time, or at different times. Depending on their location and configuration, the one or more external devices can communicate with the implantable motor disorder stimulator 132 via the RF interface circuitry 140 and/or the wireless transceiver 150.

The use of sensors to detect kinematic data is merely illustrative and other sensors could be used in accordance with embodiments presented herein. For example, the sensor 149, or another sensor, can be a sensor configured to detect/capture Electromyography (EMG) signals (e.g., muscle response or electrical activity in response to a nerve's stimulation of the muscle), Electrocochleography (ECochG) signals (e.g., measure of neuroelectric events generated by cochlear structures and the auditory nerve in response to acoustic stimulation), local field potential (LFP) signals (e.g., measure of brain activity that reflects the highly dynamic flow of information across neural networks), neurochemical dynamic signals, etc.

In certain embodiments, the external device 155 and the implantable motor disorder stimulator 132 cooperate to perform Vestibular Response Telemetry (VRT). As used herein, Vestibular Response Telemetry refers to a process in which the implanted stimulating assembly is used to detect electrically evoked compound action potentials (ECAPs) from the vestibular nerve. More specifically, once the stimulating assembly is implanted, at least one of the electrodes of the stimulating assembly is used to deliver electrical stimulation to the recipient. The ECAPs, if any, evoked by the electrical stimulation are recorded via one or more of the other implanted electrodes for subsequent analysis, display, etc. at, for example, the external device 155. ECAPs may be obtained from, or at least attempted to be obtained from, any number of the electrode contacts 156(1)-156(3). In certain embodiments, the ECAP (e.g., magnitudes) obtained from an electrode can be correlated with the effectiveness that stimulation signals delivered by that electrode will have on the vestibular nerve. Such effects can be considered during the fitting process, described below.

It is to be appreciated that the specific arrangement for implantable motor disorder stimulator 132, and more generally the system 130, shown in FIGS. 2A and 2B is merely illustrative. As such, it is to be appreciated that implantable motor disorder stimulators and associated systems may have a number of different arrangements in which, for example, the various functional components shown in FIG. 2B are implemented at one or a plurality of separate components, devices, etc.

Provided below are further details relating to: (1) the implantation of a stimulating vestibular nerve assembly of an implantable motor disorder stimulator into a recipient, (2) the “fitting” or “programming” of an implantable motor disorder stimulator for a recipient to treat motor disorders, and (3) the operation of an implantable motor disorder stimulator to electrically stimulate a recipient's vestibular system for treatment of a motor disorder. For ease of description, the techniques will primarily be described herein with reference to treatment of Parkinson's and with reference to the implantable motor disorder stimulator 132, and more generally the system 130, shown in FIGS. 2A and 2B. However, it is to be appreciated that the techniques presented herein can be applied to the treatment of other motor disorders with different system/device arrangements.

As noted above, a stimulating assembly in accordance with embodiments presented herein is configured to be implanted in the recipient so as to specifically stimulate (i.e., deliver/apply stimulation to) the recipient's vestibular system, which can include stimulation of one or more of the oval window, the sacculus, the inferior vestibular nerve, etc. In certain embodiments, the stimulating assembly is implanted adjacent the otolith organs, in particular the saccule, of the recipient's peripheral vestibular system via the recipient's oval window. From a surgical perspective, the saccule is the most interiorly (distally) accessible point of the recipient's peripheral vestibular system and is positioned immediately adjacent to the inferior branch of the vestibular nerve and near the vestibular ganglion. As such, implantation of the stimulating assembly adjacent to the saccule also places the electrodes of the stimulating assembly adjacent to the inferior branch of the vestibular nerve and the vestibular ganglion. Therefore, the positioning of the stimulating assembly adjacent to the saccule allows electrical stimulation of the inferior branch of the vestibular nerve and the vestibular ganglion that is either direct stimulation, or indirect stimulation through only the saccule. That is, the electrical stimulation (current) signals pass directly from the electrodes to the inferior branch of the vestibular nerve and/or to the vestibular ganglion, or from the electrodes to the inferior branch of the vestibular nerve and/or to the vestibular ganglion via the saccule. The positioning of the stimulating assembly adjacent to the saccule accordingly may ensure that the inferior branch of the vestibular nerve and the vestibular ganglion can be stimulated without having the stimulation pass through utricle (which if stimulated could potentially induce problems for the recipient). In general, the vestibular stimulation presented herein can be delivered from/via a stimulating assembly implanted within (inside) the inner ear, or from/via a stimulating assembly disposed at outer surface of the inner ear (e.g., implanted adjacent the inner ear).

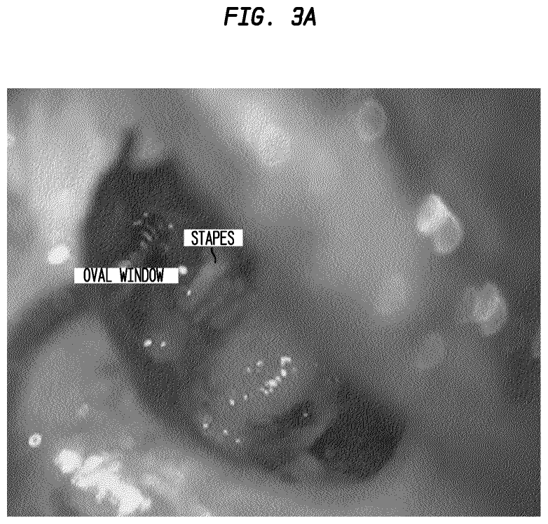

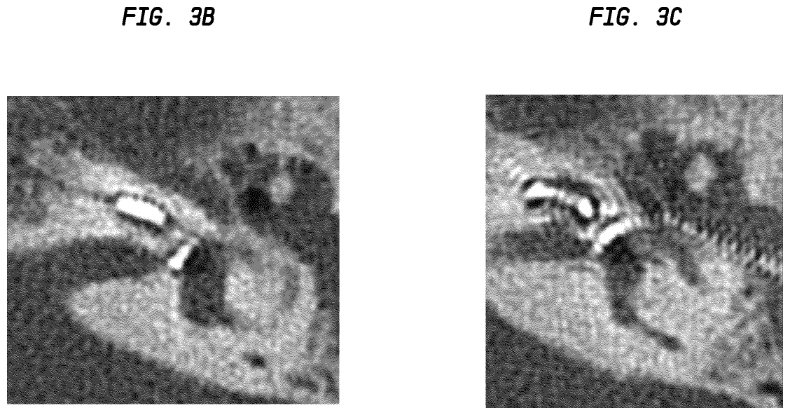

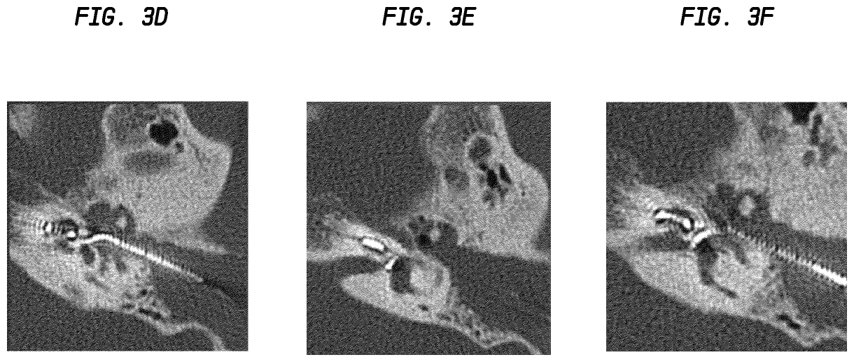

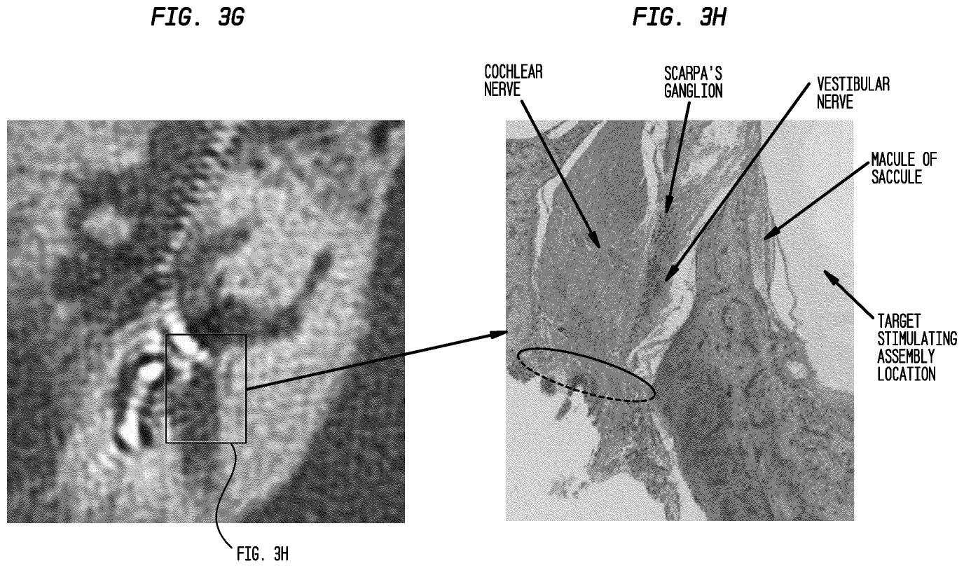

FIG. 3A is an image illustrating implantation of a stimulating assembly of a vestibular nerve stimulator into a recipient, in accordance with embodiments presented herein. More specifically, FIG. 3A illustrates insertion of a stimulating assembly via a recipient's oval window. FIGS. 3B-3G are computerized tomography (CT) scans illustrating implantation of a stimulating assembly into an inner ear of a recipient, in accordance with embodiments presented herein. As shown by FIG. 3G, and accompanying annotated medical image FIG. 3H, the stimulating assembly of a vestibular nerve stimulator in accordance with embodiments presented herein is closely positioned to the vestibular ganglion of the inferior vestibular nerve.

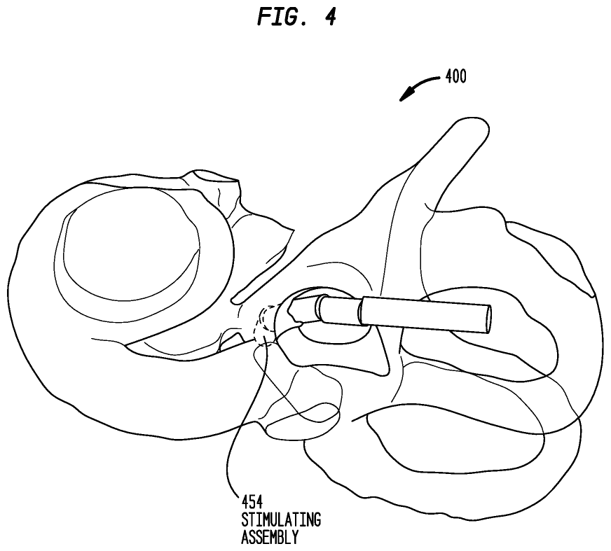

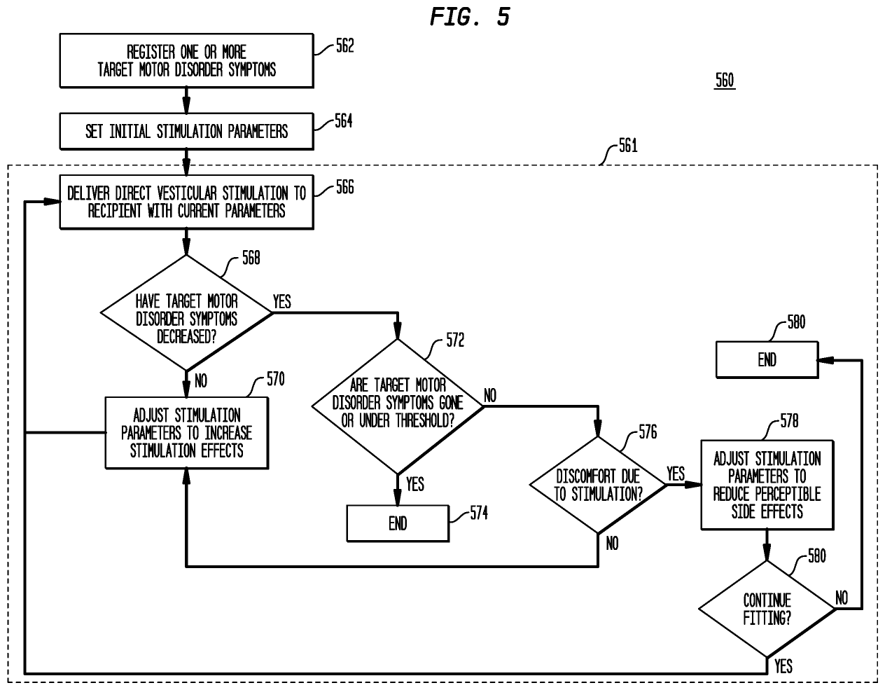

FIG. 4 is a schematic three-dimensional diagram of a recipient's inner ear 400. FIG. 4 also illustrates the general location of a stimulating assembly 454 implanted in the inner ear 400 in accordance with embodiments presented herein. In FIG. 4, the stimulating assembly 454 is positioned adjacent to the saccule so as to enable electrical stimulation of the vestibular ganglion and inferior branch of the vestibular nerve (e.g., either direct stimulation or indirect stimulation through only the saccule).

As noted, the signs and symptoms of motor disorders can vary and can include both motor symptoms (e.g., head and hand tremors, slowness of movement (e.g., bradykinesia), stiffness (e.g. rigidity), poor balance (e.g., postural instability or gait impairment), etc., and nonmotor symptoms, such as cognitive changes, constipation, excessive sweating, fatigue, hallucinations and delusions, lightheadedness, etc.

In accordance with embodiments presented herein, a recipient is diagnosed with a motor disorder (e.g., by a neurologist or other medical practitioner) and then evaluated for suitability of having an implantable motor disorder stimulator, such as implantable motor disorder stimulator 132, implanted therein. If suitable, the implantable motor disorder stimulator 132 is then implanted in the recipient and, at some point thereafter, the implantable motor disorder stimulator 132 is activated (turned on) and “fit” to the recipient.

The “fitting” of an implantable motor disorder stimulator to a recipient, sometimes also referred to as “programming” or “mapping,” is a process to determine a set of configuration settings and other data that defines the specific operational characteristics of the implantable motor disorder stimulator. In the case of implantable motor disorder stimulators presented herein, the fitting determines how the implantable motor disorder stimulator operates to deliver electrical stimulation signals (sometimes referred to herein as electrical stimulation or stimulation) to the vestibular system so to remediate symptoms of the motor disorder. That is, the fitting process is implemented to set the parameters of the electrical stimulation signal delivered to the sacculus, inferior vestibular nerve, etc. to effect a therapeutic motor benefit for the recipient, while limiting or minimizing cross-stimulation of non-target nerves, such as for example, the vagus nerve, the facial nerve, auditory nerve, etc.

In accordance with embodiments presented herein, vestibular stimulation to treat a recipient's motor disorder can take a number of different forms. For example, as described in greater detail below, the vestibular stimulation can take the form of one or more continuous pulse trains generated independent of any sensor inputs relating to motion of the head of the recipient, angular accelerations of the head, hands, etc. That is, in certain embodiments, to ensure that the recipient continually experiences relief from symptoms of the motor disorder, electrical stimulation signals can be delivered for extended periods of time, while taking into account recipient-specific characteristics and the residual effects of the stimulation. In other embodiments presented herein, the vestibular stimulation can be delivered at a specific duty cycle that ensures that the recipient continually experiences relief from symptoms of the motor disorder. In still other embodiments, the vestibular stimulation can be delivered in response to one or more sensor inputs (e.g., the vestibular stimulation is dynamically activated when one or more symptoms of the motion disorder are detected). The optimal vestibular stimulation regime (e.g., continuous stimulation, periodic stimulation, dynamic stimulation, etc.), as well as the parameters/attributes of the stimulation signals delivered to the recipient, can be determined during the fitting process. In general, the vestibular stimulation presented herein can be delivered from/via a stimulating assembly implanted within (inside) the inner ear, or from/via a stimulating assembly disposed at outer surface of the inner ear (e.g., implanted adjacent the inner ear).

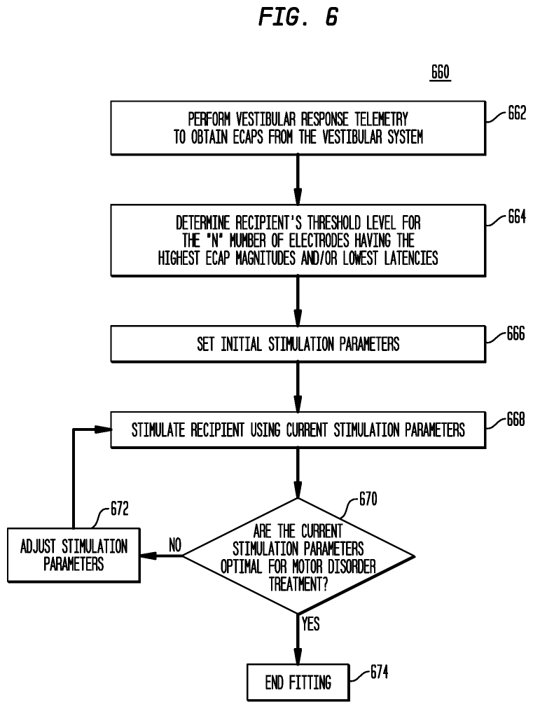

FIGS. 5 and 6 are flowcharts illustrating two example processes for fitting an implantable motor disorder stimulator to a recipient, in accordance with certain embodiments presented herein. Again, merely for ease of description, the fitting processes of FIGS. 5 and 6 will be described with reference to the implantable motor disorder stimulator 132 shown in FIGS. 2A and 2B.

Referring first to FIG. 5, shown is a fitting process/method 560. Method 560 begins at 562 where one or more subjective or objective measurements/evaluations are performed to register (e.g., identify and characterize) one or more target motor disorder symptoms of the recipient. In certain embodiments, registration of the one or more target motor disorder symptoms of the recipient can include an assessment of fine motor skills of the recipient. The fine motor skills of the recipient could be assessed, for example, via one or more handwriting tests (e.g., assessing handwriting of the recipient), an assessment of hand tremors or head tremors of the recipient, assessing a gait of the recipient, memory assessment (e.g., administration of one or more memory tests), etc. The assessment of the fine motor skills of the recipient could be based, for example, on visual observations, video graphic data, sensor data (e.g., smartwatch data, earbud data, etc.), or other information. For example, this process could utilize one or more sensors to obtain/capture one or more sensor inputs relating to kinematic data, such as linear acceleration, angular motion, or angular acceleration, of the head or hands of the recipient. In other embodiments, one or more intraoperative tests could be performed (e.g., to capture ECAP measurements from the vestibular system of the recipient) and the fine motor skills of the recipient based on the results of the one or more intraoperative tests.

In certain embodiments, registration of one or more target motor disorder symptoms of the recipient can include subjectively or objectively determining an initial baseline level for the one or more target motor symptoms of the recipient's motion, such as determining an initial baseline level of the recipient's head tremors, an initial baseline of the recipient's hand tremors, a gait analysis to determine an initial baseline level of the recipient's gait, etc. In certain embodiments, the registration of the one or more objective measurements the recipient's motion disorder can include obtaining objective measurements from the recipient, such as VRT or other neural response measurements.

In certain embodiments, the registration of the one or more target motor disorder symptoms of the recipient's motion disorder can be based on data captured by sensors to detect/capture Electromyography (EMG) signals (e.g., muscle response or electrical activity in response to a nerve's stimulation of the muscle), Electrocochleography (ECochG) signals (e.g., measure of neuroelectric events generated by cochlear structures and the auditory nerve in response to acoustic stimulation), local field potential (LFP) signals (e.g., measure of brain activity that reflects the highly dynamic flow of information across neural networks), neurochemical dynamic signals, etc.

At 564, initial stimulation parameters are set for use in delivering stimulation signals to the vestibular system via one or more of the electrode contacts 156(1)-156(3). The initial stimulation parameters can include, for example, the stimulation level (current level) for the stimulation signals, the dynamic range of the stimulation signals (e.g., threshold level and comfort level), stimulating signal timing, etc. For example, in one illustrative embodiment, the dynamic range can be set at 1 current level (CL) and the comfort level is set at 70% of threshold Vestibular Response Telemetry (VRT), if known. If no VRT is available, then the comfort level could be set at a predetermined value (e.g., 100 CL).

Although not shown in FIG. 5, in certain embodiments the operations of 564 and/or 562 can optionally be preceded by one or more diagnostic operations. These diagnostic operations can include, for example, electrode contact impedance checks, one or more VRT measurements, etc.

Returning to FIG. 5, at 566, vestibular stimulation is delivered to the recipient (e.g., electrical stimulation signals are delivered directly to the recipient's vestibular system using or more of the electrode contacts 156(1)-156(3) of the implantable motor disorder stimulator 132. Initially, the parameters of the electrical stimulation signals are those selected at 564. However, as described further below, the stimulation parameters can be adjusted/changed during the fitting process to optimize the vestibular stimulation for treatment of the recipient's motor disorder. As noted, the vestibular stimulation can be delivered from/via a stimulating assembly implanted within (inside) the inner ear, or from/via a stimulating assembly disposed at outer surface of the inner ear (e.g., implanted adjacent the inner ear).

At 568, further analysis/evaluation of the recipient's motor disorder, subsequent to the initiation of the delivery of the vestibular stimulation, is performed. That is, the one or more target motor disorder symptoms following stimulation are evaluated relative to the one or more target motor disorder symptoms prior to stimulation (e.g., re-check head or hand tremors, re-check hand writing, re-check memory, etc. to see if there is improvement in response to the stimulation). In certain embodiments, this further analysis/evaluation can include the same or similar assessments as described at 562 to register (e.g., identify and characterize) a “present” or “instant” level of the one or more target motor disorder symptoms. Thereafter, the present level of the one or more target motor disorder symptoms is evaluated relative to (e.g., compared to) the baseline level of the one or more target motor disorder symptoms. The operations at 568 can include an objective or subjective evaluation of the one or more target motor symptoms, such as an objective or subjective evaluation of whether the recipient's head or hand tremors have improved relative to the initial baseline level, an objective or subjective evaluation of whether the recipient's gait has improved relative to the initial baseline level, analysis of further VRT or other neural response measurements, etc.

As noted above, vestibular stimulation to treat a recipient's motor disorder can take a number of different form, including continuous stimulation, periodic stimulation, dynamic stimulation, etc. As such, the phrase “subsequent to the initiation of simulation” can include determinations made in conjunction with any of these or other stimulation regimes, such as a determination made while stimulation signals are being delivered to the recipient, a determination made following delivery of stimulation signals to the recipient, etc.

Returning to the example of FIG. 5, if it is determined at 568 that the one or more target motor disorder symptoms have not decreased, then the method proceeds to 570 where the stimulation parameters (e.g., current level, frequency, pulse rate, pulse gap, etc.) are adjusted to increase the effects of the vestibular stimulation. This adjustment can include, for example, increasing the current level of the stimulation signals, increasing the number of electrode contacts used to deliver the stimulation signals, changing which electrode contacts are used to deliver the stimulation, adjusting a timing of the stimulation signals, etc. After adjustment of the stimulation parameters, the method 560 then returns to 566 where vestibular stimulation is delivered to the recipient using the adjusted stimulation parameters.

Returning to 568, if it is determined that the one or more target motor disorder symptoms have decreased, then the method proceeds to 572 where a secondary determination is made as to whether the one or more target motor disorder symptoms have disappeared or are below a threshold level associated with the symptom (e.g., whether the head or hand tremors have disappeared and/or have been reduced to an acceptable level). If it is determined at 572 that the one or more target motor disorder symptoms have disappeared or are below a threshold level, then method 560 ends at 574. Method 560 reaching 574 means that the one or more target motor disorder symptoms disappeared or are below a threshold level, and no negative side effects of the stimulation, as described below, have been identified. In alternative embodiments, 574 can be replaced with one or more operational loops to further refine/optimize the stimulation signals for the recipient (e.g., fine tuning of the stimulation parameters).

Returning to 572, if it is determined that the one or more target motor disorder symptoms have not disappeared and/or are not below the threshold level, then method 560 further proceeds to 576 where another determination is made as to whether the recipient is experiencing perceptible side effects (e.g., discomfort) as a result of the vestibular stimulation. The determination of whether the recipient is experiencing side effects can include a determination of whether the recipient is experiencing any side effects, experiencing specific side effects, experiencing side effects that exceed a threshold level, etc. A number of different side effects can be evaluated at 576. In one specific example, the operations at 576 can include a determination of whether the vestibular stimulation has affected non-target areas (e.g., the vagus nerve, the facial nerve, auditory nerve, etc.) of the recipient, whether the vestibular stimulation has affected the recipient's balance, etc.

If it is determined at 576 that the recipient is not experiencing perceptible side effects, then the method proceeds to 570 where the stimulation parameters are adjusted to increase the effects of the vestibular stimulation. As noted above, this adjustment can include, for example, increasing the current level of the stimulation signals, increasing the number of electrode contacts used to deliver the stimulation signals, adjusting a timing of the stimulation signals, etc. Thereafter, the method 560 then returns to 566 where vestibular stimulation is delivered to the recipient using the adjusted stimulation parameters.

Returning to 576, if it is determined that recipient is experiencing perceptible side effects, then the method proceeds to 578 where the stimulation parameters (e.g., current level, frequency, pulse rate, pulse gap, etc.) to reduce the perceptible effects of the vestibular stimulation. These adjustment can include, for example, decreasing the current level of the stimulation signals, changing which electrode contacts are used to deliver the stimulation, decreasing the number of electrode contacts used to deliver the stimulation signals, adjusting a timing of the stimulation signals, etc.

Thereafter, the method 560 proceeds to 580 where a determination is made as to whether or not the fitting process should be continued, given the recipient has experienced perceptible side effects as a result of vestibular stimulation. This determination can be based, for example, on whether there is an expectation that the adjusted stimulation parameters (at 578) have eliminated and/or reduced the perceptible side effects. If it is determined at 580 that the fitting process can continue, then method 560 returns to 566 where vestibular stimulation is delivered to the recipient using the adjusted stimulation parameters. However, if it is determined at 580 that the fitting process cannot continue, then method 560 ends at 582. Method 560 reaching 582 means that the one or more target motor disorder symptoms have not disappeared and/or are not below a threshold level, and that one or more negative side effects of the stimulation have been identified. This is an undesirable result and would result a restart to the fitting process using new parameters, a new stimulation regime, etc.

In certain embodiments, the operations of 566, 568, 570, 572, 576, 578, and/or 580 can be operated in a closed-loop (e.g., in an automated closed loop) that continues until the method reaches 574 or 580. This closed-loop function is generally represented in FIG. 5 by dashed box 561.

It is to be appreciated that the order of operations of method 560 shown in FIG. 5 are merely illustrative and that, in certain embodiments, various operations could be performed in a different order than as shown and described. It is also to be appreciated that certain operations could be added to, or omitted from, method 560 in accordance with alternative embodiments

As noted, FIG. 6 is a flowchart illustrating another example fitting method 660, in accordance with certain embodiments presented herein. It is to be appreciated that the methods 560 and 660 are not mutually exclusive and generally illustrative complementary and/or overlapping techniques for fitting an implantable motor disorder stimulator, such as implantable motor disorder stimulator 132, to a recipient.

Method 660 begins at 662 where the electrodes 156(1)-156(3) of vestibular nerve stimulation assembly 154, when implanted in the recipient, are used for Vestibular Response Telemetry (VRT), resulting in the capture of ECAPs from the recipient's vestibular system. At 664, the captured ECAPs are used to determine the threshold (minimum) stimulation/current level (CL) that will generate an ECAP for one or more of the electrodes 156(1)-156(3). The minimum current level that will generate an ECAP for an electrode is referred to as the recipient's “threshold level” for that electrode. The recipient's threshold levels may be the same or different for each of the electrodes 156(1)-156(3).

In certain examples, the recipient's threshold level is determined for the “N” number of electrodes 156 having the highest ECAP magnitudes and/or lowest latencies (per the Vestibular Response Telemetry). In such examples, these N electrodes are the electrodes that will be used to deliver the electrical stimulation to the recipient. In one specific example, N=3 (i.e., the 3 electrodes with the highest ECAP magnitudes and/or lowest latencies are selected for use in stimulating the inferior branch of the vestibular nerve). The use of three electrodes may provide superior performance to the use of one or two electrodes (e.g., N=1 or N=2). For example, the use of three electrodes may stimulate more of the inferior branch of the vestibular nerve than the use of one or two electrodes (e.g., due to increased spread of excitation using three electrodes). If needed, non-activated electrodes 156 are deactivated by setting the current level to zero.

It is to be appreciated that operations described above with reference to 662 and 664 are optional. That is, the use of Vestibular Response Telemetry to obtain ECAPs and to determine the recipient's threshold level based thereon, may be beneficial in identifying current levels for use at the beginning of the fitting process, which may shorten the fitting process. However, the beginning current levels can also be estimated, although such estimation may lead to a longer fitting session.

Returning to the example of FIG. 6, at 666 initial stimulation parameters are is set for use in delivering stimulation signals to the vestibular system via one or more of the electrode contacts 156(1)-156(3). The initial stimulation parameters can include, for example, the dynamic range of the stimulation signals (e.g., threshold level and comfort level), stimulating timing, etc. The initial stimulation parameters may be the same or different for each of the electrode contacts 156(1)-156(3).

After the initial stimulation parameters are set, at 668 the implantable motor disorder stimulator 132 is activated (i.e., turned on) and the implantable motor disorder stimulator 132 is used to stimulate the recipient's vestibular system. At 668, the vestibular stimulation is delivered in accordance with the “current” (currently instantiated) stimulation parameters, which initially comprise the initial vestibular stimulation parameters.

At 670, one or more determinations are made as whether or not the current stimulation parameters are optimal for treatment of the recipient's disorder. In certain embodiments, the or more determinations at 670 can include an objective or subjective evaluation of one or more specific or target motor symptoms of the recipient's motor disorder (target motor disorder symptoms), such as an objective or subjective evaluation of whether the recipient's head or hand tremors have improved relative to the initial baseline level, tremors, an objective or subjective evaluation of whether the recipient's gait has improved relative to the baseline level, analysis of further VRT or other neural response measurements, etc. The or more determinations at 670 can also include an objective or subjective evaluation of whether or not tmade as to whether the one or more target motor disorder symptoms have disappeared or are below a threshold level associated with the symptom (e.g., whether the head or hand tremors have disappeared and/or have been reduced to an acceptable level). Moreover, the or more determinations at 670 can also or alternatively include a determination of whether the recipient is experiencing any perceptible side effects, which may include a determination of whether the recipient is experiencing specific side effects, experiencing side effects that exceed a threshold level, etc. A number of different side effects, such as whether the vestibular stimulation has affected the facial nerve of the recipient, can be evaluated at 670.

As noted above, the phrase “subsequent to the initiation of simulation” can include determinations made in conjunction with any of these or other stimulation regimes, such as a determination made while stimulation signals are being delivered to the recipient, a determination made following delivery of stimulation signals to the recipient, etc.

If it is determined at 670 that the current stimulation parameters are non-optimal (e.g., the recipient one or more target motor disorder symptoms have not disappeared and/or are not below a threshold level associated with the symptom, or the recipient is experiencing perceptible side effects), then method 660 proceeds to 672. At 672, the stimulation parameters are adjusted. For example, the current level of stimulation signals delivered via one or more of the electrodes may be increased (e.g., +2), the pulse rate could be changed, etc. Once selected, the adjusted stimulation parameters are instantiated in the implantable motor disorder stimulator 132 and become the “current” vestibular stimulation parameters.

After selection and instantiation of the adjusted stimulation parameters as the current vestibular stimulation parameters, method 660 returns to 668 where the adjusted stimulation parameters are used to stimulate the recipient's vestibular system. The operations at 670, 672, and 668 are then iteratively repeated until a determination is made at 670 that the current stimulation parameters are optimal for treatment of the recipient's motor disorder. Once it is determined at 670 that the current vestibular stimulation parameters are optimal, the method 660 ends at 674.

As noted above, the use of Vestibular Response Telemetry to obtain ECAPs, and determining the recipient's threshold level based thereon, may be beneficial for fitting speeds up the fitting process (i.e., the threshold levels provide a good starting point for the fitting procedure, which means the time taken for the fitting process will be much shorter). However, also as noted, the use of Vestibular Response Telemetry during fitting is optional and the fitting may instead being some estimated current levels.

In certain examples, the ECAPs (e.g., magnitudes) obtained from an electrode can be correlated with the effectiveness that stimulation signals delivered by that electrode will have on the vestibular nerve. These effects can be considered during the fitting process of FIG. 6. For example, no ECAP is obtained from an implanted electrode (or the obtain ECAP is very low), then that electrode may be excluded from use in delivering stimulation signals to the vestibular nerve, the levels of the stimulation signals delivered from that electrode may be reduced, etc.

It is to be appreciated that the order of operations of method 660 shown in FIG. 6 are merely illustrative and that, in certain embodiments, various operations could be performed in a different order than as shown and described. It is also to be appreciated that certain operations could be added to, or omitted from, method 660 in accordance with alternative embodiments.

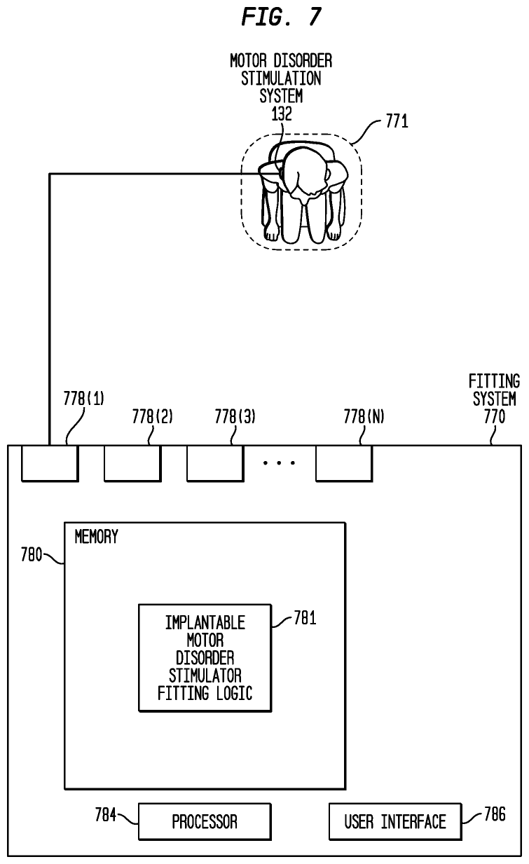

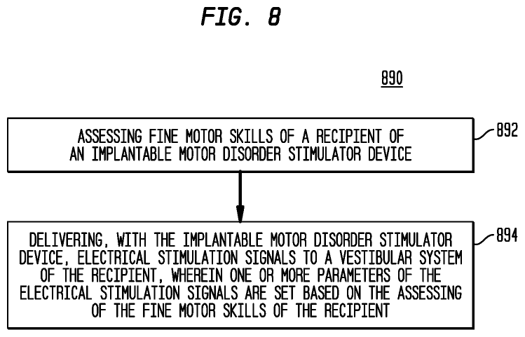

Shown in FIG. 7 is a block diagram illustrating an example fitting system 770 configured to execute aspects of the techniques presented herein. Fitting system 770 is, in general, a computing device that comprises a plurality of interfaces/ports 778(1)-778(N), a memory 780, a processor 784, and a user interface 786. The interfaces 778(1)-778(N) may comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 7, interface 778(1) is configured to communicate with the motor disorder stimulation system 130 having components implanted in a recipient 771. For example, interface 778(1) can directly communicate with the implantable motor disorder stimulator 132 (e.g., via wireless transceiver 150), or communicate with the implantable motor disorder stimulator 132 via the external device 155 that, as noted above, is in communication with the implantable motor disorder stimulator 132. Interface 778(1) may be configured to communicate via a wired or wireless connection (e.g., telemetry, Bluetooth, etc.).

The user interface 786 includes one or more output devices, such as a display screen (e.g., a liquid crystal display (LCD)) and a speaker, for presentation of visual or audible information to a clinician, audiologist, or other user. The user interface 786 may also comprise one or more input devices that include, for example, a keypad, keyboard, mouse, touchscreen, etc.

The memory 780 comprises implantable motor disorder stimulator fitting logic 781 that may be executed to fit an implantable motor disorder stimulator. For example, the implantable motor disorder stimulator fitting logic 781 can be executed by processor 784 to perform aspects of methods 560 and 660, described above, or other methods in accordance with the fitting techniques presented herein.

Memory 780 may comprise read only memory (ROM), random access memory (RAM), magnetic disk storage media devices, optical storage media devices, flash memory devices, electrical, optical, or other physical/tangible memory storage devices. The processor 784 is, for example, a microprocessor or microcontroller that executes instructions for the implantable motor disorder stimulator fitting logic 781. Thus, in general, the memory 780 may comprise one or more tangible (non-transitory) computer readable storage media (e.g., a memory device) encoded with software comprising computer executable instructions and when the software is executed (by the processor 784) it is operable to perform the techniques described herein.

As noted above, FIG. 7 illustrates an example fitting system, while FIGS. 5 and 6 illustrate two example methods for fitting methods, for use with an implantable motor disorder stimulator, such as implantable motor disorder stimulator 132. The fitting of the implantable motor disorder stimulator 132 can occur shortly after initial implantation, but can also be performed regularly/periodically thereafter to ensure that the stimulation remains remain optimal for the current stage of the recipient's motor disorder. That is, as noted, most motor disorders are progressive and the recipient's symptoms will become worse over time. As such, the above fitting methods, or modified versions thereof, could be conducted on a regular or periodic basis to adjust the stimulation settings to account for the motor disorder progression.

After the implantable motor disorder stimulator 132 is implanted and fit to the recipient, the implantable motor disorder stimulator will operate, in real-time, to electrically stimulate the recipient's vestibular system (e.g., sacculus, inferior vestibular nerve, oval window, etc.) so as to remediate symptoms of the recipient's motor disorder. For example, in the context of Parkinson's, the implantable motor disorder stimulator 132 can be fit to the recipient so as to reduce, minimize, or potentially eliminate head tremors, hand tremors, improve gait, etc.

As described above, vestibular stimulation to treat a recipient's motor disorder can take a number of different forms, including continuous stimulation, periodic stimulation, dynamic stimulation, etc. The optimal vestibular stimulation regime (e.g., continuous stimulation, periodic stimulation, dynamic stimulation, etc.), as well as the parameters/attributes of the stimulation signals delivered to the recipient, can be determined during a fitting process, as described above.

As noted, in certain embodiments, the vestibular stimulation can take the form of one or more continuous pulse trains generated independent of any sensor inputs relating to motion of the head of the recipient, angular accelerations of the head, hands, etc. That is, in certain embodiments, to ensure that the recipient continually experiences relief from symptoms of the motor disorder, electrical stimulation signals can be delivered the vestibular system for extended periods of time, while taking into account recipient-specific characteristics and the residual effects of the stimulation. In certain such examples, the electrical stimulation signals are delivered to the recipient continually constantly through the day (e.g., continually deliver stimulation signals for 8 hours, 12 hours, 14 hours, etc.).

As noted, an implantable motor disorder stimulator in accordance with embodiments presented generally includes one or more electrode contacts (e.g., three electrode contacts, five electrode contacts, etc.) implanted in the recipient adjacent to the vestibular system. In examples in which a plurality of electrode contacts are implanted, one or all of the electrode contacts may be used to deliver vestibular stimulation to the recipient and the stimulation may be the same or different for different electrodes. For example, multiple electrodes may deliver pulse trains that are generated using the same fixed stimulation parameters. Alternatively, an implantable motor disorder stimulator may deliver at least a first continuous pulse train to a first one of the plurality of stimulating elements and deliver at least a second continuous pulse train to a second one of the plurality of stimulating elements, where the first and second pulse trains are generated in accordance with different stimulation parameters (e.g., different current levels, different pulse rates, etc.). Again, the different stimulation parameters for the different pulse trains are determined during the fitting process, where the differences may be based on the electrode positioning, ECAP responses, subjective recipient feedback, etc.

As noted above, the implantable motor disorder stimulator is configured to remediate motor symptoms associated with a motion disorder. In other words, an implantable motor disorder stimulator electrically stimulates the recipient's vestibular nerve (e.g., the inferior branch of the vestibular nerve and/or the vestibular ganglion) in a manner (e.g., timing, stimulation parameters, etc.) that improves, e.g., stabilizes, the recipient's motor function. In these embodiments, the stimulation signals are generated and delivered independent of any sensor data.

As noted, in accordance with embodiments presented herein, the electrical stimulation signals are delivered so that the recipient continually experiences relief from one or more motor symptoms of a motor disorder. Also as noted above, in certain embodiments, the vestibular stimulation is delivered for extended periods of time, while taking into account recipient-specific characteristics and the residual effects of the stimulation. In certain examples, the electrical stimulation signals are delivered to the recipient continually constantly through the day (e.g., continually deliver stimulation signals for 8 hours, 12 hours, 14 hours, etc.).

For certain recipients, the vestibular stimulation may produce residual effects for some period of time after cessation of the stimulation. These residual effects may be leveraged in certain embodiments to implement periodic stimulation patterns (e.g., deliver vestibular stimulation at a specific duty cycle that ensures that the recipient continually experiences relief from symptoms of the motor disorder). For example, an implantable motor disorder stimulator in accordance with embodiments presented herein could deliver stimulation signals to the vestibular system at a specific duty cycle over the course of the day or use of the device. In one example, an implantable motor disorder stimulator could operate at a fifty (50) percent duty cycle (e.g., continuously deliver stimulation signals for 30 minutes, followed by no stimulation for the following 30 minutes), a forty (40) percent duty cycle (e.g., continuously deliver stimulation signals for 24 minutes, followed by no stimulation for the following 36 minutes), a sixty (60) percent duty cycle (e.g., continuously deliver stimulation signals for 36 minutes, followed by no stimulation for the following 24 minutes), and so on. The use of such duty cycles may be possible due to residual effects of the stimulation signals in the vestibular system for some period of time after cessation of the stimulation.

The residual effects of vestibular stimulation may be different for different recipients, thus the selected duty cycles may be different for different recipients (e.g., recipient-specific) and could be determined during the fitting process. However, as noted above, implantable motor disorder stimulators in accordance with embodiments presented herein are configured to treat chronic motor disorders and, accordingly, electrical stimulation signals so that the recipient continually experiences relief from motor disorder systems. Therefore, the duty cycles are selected so that the stimulator will deliver stimulation signals before the residual effects of prior stimulation cease. For example, if a recipient's residual effects last 60 minutes, the vestibular stimulation may use a duty cycle where the device only pauses stimulation for 45 minutes. Therefore, the selected duty cycles (i.e., pauses in stimulation) are based on the recipient-specific information, such as their personal residual effects to vestibular nerve stimulation.

In still other embodiments, the vestibular stimulation can be delivered in response to one or more sensor inputs (e.g., the vestibular stimulation is dynamically activated when one or more symptoms of the motion disorder are detected). For example, as noted above, the implantable motor disorder stimulator 132 could include one or more sensors 149 and/or the implantable motor disorder stimulator 132 could communicate with an external device (e.g., mobile phone, smart watch, etc.) that includes one or more sensors. In these embodiments, the sensors could be used to detect symptoms of a motor disorder, such as head tremors or hand tremors, and generate sensor data representing attributes of the detected symptoms.

In one example dynamic activation arrangement, one or more sensors could detect head or hand tremors, and provide sensor data representing an amount of tremor detect (e.g., small amount of tremor, large amount of tremor, tremor exceeding a threshold level, etc.). The information included in the sensor data could be used to activate/control delivery of vestibular stimulation via the implantable motor disorder stimulator 132. This activation can include dynamically generating stimulation signals based on the content of the sensor data, activating one or more previously determined stimulation programs (e.g., initiate delivery of continuous stimulation for a period of time), etc. As such, in certain embodiments, the motor disorder stimulation system 130 operates in a closed-loop to detect motor disorder symptoms and activate vestibular stimulation to remediate the detected motor disorder symptoms, including real-time stimulation parameter adjustments based on attributes of the motor disorder symptoms experienced by the recipient.

Current treatments for motor disorders, such as Parkinson's, include drug treatments or therapies. Drug treatments or therapies may involve, for example, the administration of a dopamine precursor that is converted to dopamine within the central nervous system (i.e., Levodopa (L-dopa)). Other types of drug therapies are also available. Unfortunately, drug therapies frequently become less effective or ineffective over time for an undesirably large population of Parkinson's patients. For example, a Parkinson's patient may require multiple drugs in combination to extend the time period of efficacy of drug therapies. Drug treatments additionally have a significant likelihood of inducing undesirable physical side effects; motor function complications such as uncontrollable involuntary movements (dyskinesias) are a particularly common side effect.

In accordance with certain embodiments presented herein, the vestibular stimulation can be used in combination with drug treatments or therapies in order to treat a recipient's motor disorder. For example, as noted, in the context of Parkinson's, certain drug treatments are intended to trigger the generation of dopamine. The vestibular stimulation techniques presented herein may also to trigger the generation of dopamine. As a result, the combination of vestibular stimulation and drug treatment could, for example, reduce the required drug dosage and, potentially, extend the period of time over which the drug treatment remains effective/provide a longer period of time over which the drug dosage could be increased. Conversely, the combination of vestibular stimulation and drug treatment could enable lower use of lower current levels for vestibular stimulation to achieve a therapeutic effect.

FIG. 8 is a flowchart of a method 890 in accordance with embodiments presented herein. Method 890 begins at 892 where fine motor skills of a recipient of an implantable motor disorder stimulator device are assessed. At 894, the implantable motor disorder stimulator device delivers electrical stimulation signals to a vestibular system of the recipient. One or more parameters of the electrical stimulation signals are set based on the assessing of the fine motor skills of the recipient.

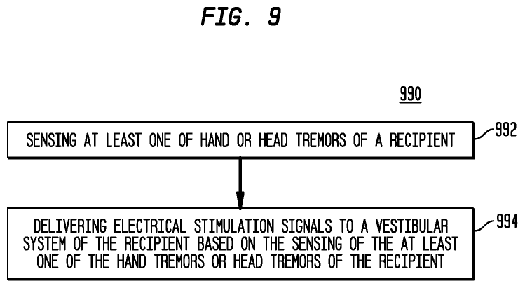

FIG. 9 is a flowchart of a method 990 in accordance with embodiments presented herein. Method 990 begins at 992 where at least one of hand or head tremors of a recipient are sensed. At 994, electrical stimulation signals are delivered to a vestibular system of the recipient based on the sensing of the at least one of the hand tremors or head tremors of the recipient.

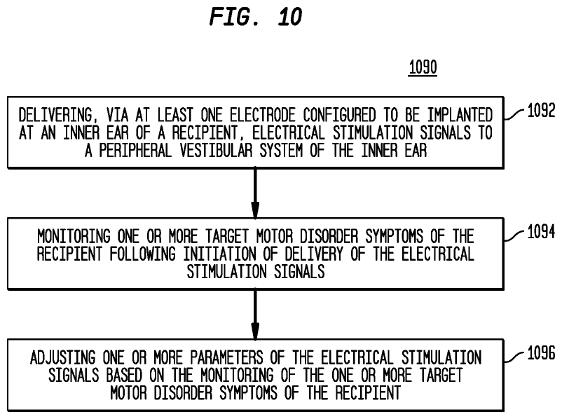

FIG. 10 is a flowchart of a method 1090 in accordance with embodiments presented herein. Method 1090 begins at 1092 where electrical stimulation signals are delivered, via at least one electrode configured to be implanted at an inner ear of a recipient, to a peripheral vestibular system of the inner ear. At 1094, one or more target motor disorder symptoms of the recipient are monitored following initiation of delivery of the electrical stimulation signals. At 1096, one or more parameters of the electrical stimulation signals are adjusted based on the monitoring of the one or more target motor disorder symptoms of the recipient.

It is to be appreciated that the above described embodiments are not mutually exclusive and that the various embodiments can be combined in various manners and arrangements.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method, comprising:
assessing fine motor skills of a recipient of an implantable motor disorder stimulator device; and
delivering, with the implantable motor disorder stimulator device, electrical stimulation signals to a vestibular system of the recipient,
wherein one or more parameters of the electrical stimulation signals are set based on the assessing of the fine motor skills of the recipient.

2. The method of claim 1, wherein assessing the fine motor skills of the recipient includes assessing handwriting of the recipient.

3. The method of claim 1, wherein assessing the fine motor skills of the recipient includes assessing at least one of hand tremors or head tremors of the recipient.

4. The method of claim 3, further comprising:
capturing one or more sensor inputs relating to linear acceleration, angular motion, or angular acceleration of the head of the recipient.

5. The method of claim 3, further comprising:
capturing one or more sensor inputs relating to linear acceleration, angular motion, or angular acceleration of a hand of the recipient.

6. The method of claim 1, wherein assessing the fine motor skills of the recipient includes assessing a gait of the recipient.

7. The method of claim 1, wherein assessing the fine motor skills of the recipient includes:
performing one or more intraoperative tests; and
assessing the fine motor skills of the recipient based on the one or more intraoperative tests.

8. The method of claim 7, wherein performing one or more intraoperative tests comprises:
obtaining one or more Electrically Evoked Compound Action Potential (ECAP) measurements from the vestibular system of the recipient.

9. The method of claim 1, wherein assessing the fine motor skills of the recipient includes:
confirming that the recipient suffers from a motor disorder.

10. The method of claim 9, wherein confirming that the recipient suffers from a motor disorder includes:
confirming that the recipient suffers from Parkinson's disease.

11. The method of claim 9, wherein confirming that the recipient suffers from a motor disorder:
confirming that the recipient suffers from ataxia.

12. The method of claim 1, wherein the one or more parameters of the electrical stimulation signals that are set based on the assessment of the fine motor skills of the recipient include one or more of current level, frequency, pulse rate, or pulse gap, of the electrical stimulation signals.

13. The method of claim 1, further comprising:
delivering the electrical stimulation signals independent of any sensor inputs relating to linear acceleration, angular motion, or angular acceleration of the head or hands of the recipient.

14. The method of claim 13, wherein delivering the electrical stimulation signals further comprises:
delivering at least a first continuous pulse train via at least a first one of a plurality of stimulating elements configured to be implanted at an inner ear of the recipient.

15. The method of claim 14, wherein delivering the electrical stimulation signals further comprises:
delivering at least a first continuous pulse train via at least a first one of a plurality of stimulating elements configured to be implanted in the inner ear of the recipient.

16. The method of claim 1, wherein delivering the electrical stimulation signals to a vestibular system of the recipient comprises:
delivering the electrical stimulation signals to at least the inferior branch of the vestibular nerve through one or more of the otolith organs.

17-30. (canceled)

31. One or more non-transitory computer readable storage media comprising instructions that, when executed by a processor, cause the processor to:
deliver, via at least one electrode configured to be implanted at an inner ear of a recipient, electrical stimulation signals to a peripheral vestibular system of the inner ear;
monitor one or more target motor disorder symptoms of the recipient following initiation of delivery of the electrical stimulation signals; and
adjust one or more parameters of the electrical stimulation signals based on the monitoring of the one or more target motor disorder symptoms of the recipient.

32. The one or more non-transitory computer readable storage media of claim 31, further comprising instructions operable to:
prior to delivering the electrical stimulation signals, register the one or more target motor disorder symptoms of the recipient.

33. The one or more non-transitory computer readable storage media of claim 32, wherein the instructions operable to register the one or more target motor disorder symptoms of the recipient comprise instructions operable to:
determine a baseline level for the one or more target motor disorder symptoms of the recipient.

34. The one or more non-transitory computer readable storage media of claim 33, wherein the instructions operable to monitor one or more target motor disorder symptoms of the recipient following initiation of delivery of the electrical stimulation signals comprise instructions operable to:
determine a present level for the one or more target motor disorder symptoms of the recipient; and
compare the present level for the one or more target motor disorder symptoms to the baseline level for the one or more target motor disorder symptoms.

35. The one or more non-transitory computer readable storage media of claim 31, further comprising instructions operable to:
monitor the recipient to determine whether the electrical stimulation signals have crossstimulated one or more non-target nerve areas.

36. The one or more non-transitory computer readable storage media of claim 35, further comprising instructions operable to:
determine that the electrical stimulation signals have affected one or more non-target areas; and
adjust the electrical stimulation signals to reduce cross-stimulation of the one or more non-target nerve areas.

37. The one or more non-transitory computer readable storage media of claim 31, wherein the instructions operable to monitor one or more target motor disorder symptoms of the recipient following initiation of delivery of the electrical stimulation signals comprise instructions operable to:
monitor at least one of hand tremors or head tremors of the recipient.

38. The one or more non-transitory computer readable storage media of claim 37, further comprising instructions operable to:
capture one or more sensor inputs relating to linear acceleration, angular motion, or angular acceleration of at least one of the head or hands of the recipient.

39. The one or more non-transitory computer readable storage media of claim 31, wherein the instructions operable to monitor one or more target motor disorder symptoms of the recipient following initiation of delivery of the electrical stimulation signals comprise instructions operable to:
monitor a gait of the recipient.

40. The one or more non-transitory computer readable storage media of claim 31, wherein the instructions operable to adjust one or more parameters of the electrical stimulation signals based on the monitoring of the one or more target motor disorder symptoms of the recipient comprise instructions operable to:
adjust a level of the electrical stimulation signals.

41. The one or more non-transitory computer readable storage media of claim 31, wherein the at least one electrode comprises a plurality of stimulating elements, and wherein the instructions operable to adjust one or more parameters of the electrical stimulation signals based on the monitoring of the one or more target motor disorder symptoms of the recipient comprise instructions operable to:
change which of the plurality of stimulating elements are used to deliver the electrical stimulation signals.

42. The one or more non-transitory computer readable storage media of claim 31, wherein the instructions operable to deliver the electrical stimulation signals to the peripheral vestibular system of the inner ear comprise instructions operable to:
deliver at least a first continuous pulse train via the at least one electrode.

43. The one or more non-transitory computer readable storage media of claim 31, wherein the instructions operable to deliver the electrical stimulation signals to the peripheral vestibular system of the inner ear comprise instructions operable to:
deliver the electrical stimulation signals to at least the inferior branch of the vestibular nerve through one or more of the otolith organs.

44. (canceled)

45. (canceled)

46. An implantable motor disorder stimulator system, comprising:
one or more electrodes configured to be implanted at an inner ear of a recipient with a motor disorder;
an implantable stimulator unit configured to deliver, via at least one of the one or more electrodes, electrical stimulation signals to a vestibular system of the inner ear of the recipient; and
at least one processor configured to:
following delivery of the electrical stimulation signals to a vestibular system, analyze one or more motor disorder symptoms experienced by the recipient, and
adjust, based on the analyzing of the one or more motor disorder symptoms experienced based on the recipient, one or more parameters of the electrical stimulation signals to reduce the one or more motor disorder symptoms experienced by the recipient.

47. The implantable motor disorder stimulator system of claim 46, where to analyze the one or more motor disorder symptoms experienced by the recipient the at least one processor is configured to:
receive an output from one or sensors relating to at least one of hand or head tremors of the recipient; and
analyze the at least one of the hand or head tremors of the recipient.

48. The implantable motor disorder stimulator system of claim 47, wherein to adjust the one or more parameters of the electrical stimulation signals to reduce the one or more motor disorder symptoms experienced by the recipient, the at least one processor is configured to:
adjust the electrical stimulation signals to reduce the at least one of the hand tremors or head tremors of the recipient.

49. The implantable motor disorder stimulator system of claim 46, wherein to adjust the one or more parameters of the electrical stimulation signals to reduce the one or more motor disorder symptoms experienced by the recipient, the at least one processor is configured to:
activate a predetermined stimulation sequence in response to sensing at least one of hand tremors or head tremors of the recipient.

50. The implantable motor disorder stimulator system of claim 49, wherein to activate a predetermined stimulation sequence, the at least one processor is configured to:
activate a stimulation sequence comprising one or more continuous electrical pulse trains.

51. The implantable motor disorder stimulator system of claim 50, wherein the at least one processor is configured to:
deliver the one or more continuous electrical pulse trains for period of time.

52. The implantable motor disorder stimulator system of claim 46, wherein the at least one processor is configured to:
monitor at least one of hand or head tremors of the recipient;
determine that the at least one of the hand or head tremors of the recipient are below a threshold level; and
adjust the one or more parameters of the electrical stimulation signals following determining that the at least one of the hand or head tremors of the recipient are below the threshold level.

53. (canceled)
